(19) United States
(12) Patent Application Publication
(10) Pub. No.: US 2007/0073390 A1
(43) Pub. Date: Mar. 29, 2007

(54) Methods and devices for enhanced adhesion between metallic substrates and bioactive material-containing coatings

(75) Inventor: Michael J. Lee, Santa Rosa, CA (US)

Correspondence Address: KIRKPATRICK & LOCKHART PRESTON GATES ELLIS LLP;ATTN: C. RACHAL WINGER, 925 FOURTH AVE, SUITE 2900, SEATTLE, WA, 98104-1158, US

(73) Assignee: Medlogics Device Corporation, Santa Rosa, CA (US)

(21) Appl. No.: 11/233,965

(22) Filed: Sep. 23, 2005

(51) Int. Cl. A61F 2/82

(52) U.S. Cl. 623/1.46; 623/23.57; 427/2.25; 204/471; 205/261

(57) ABSTRACT

Disclosed herein are methods to create medical devices and medical devices including bioactive composite structures with enhanced adhesion characteristics. The bioactive composite structures are prepared using anchors that are electrochemically codeposited into a metallic layer that is formed on the surface of implantable medical device followed by the adhesion of a bioactive material-containing coating to the substrate and anchors.

### FIELD OF THE INVENTION

The present invention relates to methods for providing enhanced adhesion between metallic substrates, such as implantable medical devices, and bioactive material-containing coatings. The present invention also relates to methods for providing enhanced adhesion between a metallic substrate, such as an implantable medical device and a polymeric bioactive material-containing coating.

### BACKGROUND OF THE INVENTION

In many circumstances, it is beneficial for an implanted medical device to release a bioactive material into the body once the device has been implanted. Such released bioactive materials can enhance the treatment offered by the implantable medical device, facilitate recovery in the implanted area and lessen the local physiological trauma associated with the implant.

Vascular stents are one type of device that has benefited from the inclusion of bioactive materials. Stents are ridged, or semi-ridged, tubular scaffoldings that are deployed within the lumen (inner tubular space) of a vessel or duct during angioplasty or related procedures intended to restore patency (openness) to vessel or duct lumens. Stents generally are left within the lumen of a vessel or duct after angioplasty or a related procedure to reduce the risks of the vessel renarrowing chronically (“restenosis”), closing down acutely (“abrupt closure”) or reoccluding (all of which are hereinafter referred to as “reclosure”). While stents themselves aid in the prevention of reclosure, including bioactive materials on the surface of the implanted stent can inhibit or prevent reclosure even further.

One challenge in the field of implantable medical devices has been adhering bioactive materials to the surfaces of implantable devices so that the bioactive materials will be released over time once the device is implanted. One approach to adhering bioactive materials to substrates, such as the surface of implantable medical devices has been to include the bioactive materials in polymeric coatings. Polymeric coatings can hold bioactive materials onto the surface of implantable medical devices and release the bioactive materials via degradation of the polymer or diffusion into liquid or tissue (in which case the polymer is non-degradable). While polymeric coatings can be used to adhere bioactive materials to implanted medical devices, there are problems associated with their use. One problem is that adherence of a polymeric coating to a substantially different substrate, such as a stent's metallic substrate, is difficult due to differing characteristics of the materials (such as differing thermal expansion properties). This difficulty in adhering the two different material types often leads to inadequate bonding between the medical device and the overlying polymeric coating which can result in the separation of the materials over time. Such separation is an exceptionally undesirable property in an implanted medical device.

One way to help to prevent separation of a bioactive material-containing coating from an underlying metallic substrate is to fully encapsulate the substrate within the bioactive material-containing coating. Fully encapsulating the substrate means that the bioactive material-containing coating fully covers the implantable medical device so that the coating binds to itself and “traps” the implantable medical device within its “shell.” While this approach can prevent complete separation of the two different materials, it often adds unnecessary and undesirable bulk to the implantable medical device. Therefore, a need exists for methods to adhere bioactive material-containing coatings to metallic substrates such as implantable medical devices that do not rely on full encapsulation. The present invention addresses this need.

### SUMMARY OF THE INVENTION

The present invention addresses drawbacks associated with previously-available methods of coating implantable medical devices with bioactive material-containing coatings by providing “anchors” on the surface of a metallic substrate to which bioactive material-containing coatings can bind. The anchors of the present invention are the same material or a material with substantially similar characteristics as the bioactive material-containing coating. Thus, the bioactive material-containing coating can stably bind to the anchors, diminishing the risk of separation while not relying on full encapsulation of the implantable medical device. The anchors are created on the surface of an implantable medical device by electrochemically codepositing them into a metallic layer that is formed over the surface of the implantable medical device.

Specifically, a metallic implantable medical device can have a metallic layer deposited over its surface through an electrochemical process. Because the deposited metallic layer will have similar physical properties to the underlying device, the deposited metallic layer will adhere to the surface of the implantable medical device. During deposition of this metallic layer, anchors can be codeposited with the metallic layer. Importantly, the codeposited anchors can contain bioactive materials themselves or can be bioactive material free. The only requirement placed on these anchors is that they be the same material or a material with substantially similar characteristics as the bioactive material-containing coating that will eventually be placed onto the surface of the implantable medical device.

When anchors are codeposited into an electrochemically formed metallic layer, these anchors are effectively trapped within the depositing metallic layer. A portion of the trapped anchors will be on the surface of the deposited metallic layer, providing a material with similar or identical physical characteristics to the bioactive material-containing coating that will be adhered to the surface of the implantable medical device. Thus, these exposed portions (i.e. anchors) provide a substrate to which the bioactive material-containing coating can bind with enhanced adhesion characteristics as opposed to its ability to bind to bare metal.

One embodiment of the methods of the present invention includes providing a solution comprising metal ions and anchors; contacting a substrate with the solution thereby forming a metallic composite structure through an electrochemical process wherein at least a subset of the anchors are exposed on at least a portion of the surface of the formed structure, and adhering a bioactive material-containing coating to the surface of the structure and the exposed anchors wherein the bioactive material-containing coating and the anchors have physical characteristics that are more similar than the physical characteristics of the bioactive material-containing coating and the substrate.

In another embodiment of the methods of the present invention, the anchors include a bioactive material and the formed structure is a bioactive composite structure. In another embodiment of the methods of the present invention, the anchors are free of bioactive materials and the formed structure is a composite structure.

In another embodiment of the methods of the present invention, the electrochemical process is an electrolytic codeposition process. In another embodiment of the methods of the present invention, the electrochemical process is an electroless codeposition process. In another embodiment of the methods of the present invention, the electrochemical process is an electrophoretic codeposition process.

In another embodiment of the methods of the present invention, the anchors comprise a polymer. In another embodiment of the methods of the present invention, the bioactive material-containing coating comprises a polymer. In another embodiment of the methods of the present invention, the anchors and the bioactive material-containing coating both comprise a polymer.

In another embodiment of the methods of the present invention, the anchors and bioactive material-containing coating are only applied to a portion of the substrate.

In another embodiment of the methods of the present invention, the substrate is a stent.

In another embodiment of the methods of the present invention, a topcoat is formed over the adhered bioactive material-containing coating.

In another embodiment of the methods of the present invention, before the providing of the solution and the contacting of the substrate with the solution, a strike layer is formed on the surface of the substrate. In another embodiment of the methods of the present invention, before the providing of the solution and the contacting of the substrate with the solution, a seed layer is formed on the surface of the substrate. In another embodiment of the methods of the present invention, before the providing of the solution and the contacting of the substrate with the solution, a strike layer is formed on the surface of the substrate and a seed layer is formed on the surface of the strike layer.

The present invention also includes medical devices. In one embodiment of the medical devices of the present invention, the medical device comprises a substrate having a metallic composite structure containing anchors wherein the structure is formed through an electrochemical process and wherein at least a subset of the anchors are exposed on the surface of the structure; and a bioactive material-containing coating adhered to the surface of the structure and the exposed anchors and wherein the bioactive material-containing coating and the anchors have physical characteristics that are more similar than the physical characteristics of the bioactive material-containing coating and the substrate.

In another embodiment of the medical devices of the present invention, the anchors include a bioactive material and the formed structure is a bioactive composite structure. In another medical device of the present invention, the anchors are free of bioactive materials and the formed structure is a composite structure.

In another embodiment of the medical devices of the present invention, the electrochemical process is an electrolytic codeposition process. In another embodiment of the medical devices of the present invention, the electrochemical process is an electroless codeposition process. In another embodiment of the medical devices of the present invention, the electrochemical process is and an electrophoretic codeposition process.

In another embodiment of the medical devices of the present invention, the anchors comprise a polymer. In another embodiment of the medical devices of the present invention, the bioactive material-containing coating comprises a polymer. In another embodiment of the medical devices of the present invention, the anchors and the bioactive material-containing coating both comprise a polymer.

In another embodiment of the medical devices of the present invention, the anchors and bioactive material-containing coating are only applied to a portion of the substrate.

In another embodiment of the medical devices of the present invention, the substrate is a stent.

In another embodiment of the medical devices of the present invention, the medical device comprises a topcoat over the adhered bioactive material-containing coating.

In another embodiment of the medical devices of the present invention, a strike layer is formed on the surface of the substrate. In another embodiment of the medical devices of the present invention, a seed layer is formed on the surface of the substrate. In another embodiment of the medical devices of the present invention, a strike layer is formed on the surface of the substrate and a seed layer is formed on the surface of the strike layer.

### BRIEF DESCRIPTION OF THE FIGURES

FIG. 1 depicts a fragmented cross-section of a stent of the present invention including a metallic layer with electrochemically codeposited anchors and a bioactive material-containing coating.

FIG. 2 depicts a fragmented cross-section of a stent of the present invention including a metallic layer with electrochemically codeposited anchors, a bioactive material-containing coating, a strike layer, a seed layer and a topcoat.

### DETAILED DESCRIPTION

I. Definitions

Some terms that are used herein are described as follows.

The term “bioactive material(s)” refers to any organic, inorganic, or living agent that is biologically active or relevant. For example, a bioactive material can be a protein, a polypeptide, a polysaccharide (e.g. heparin), an oligosaccharide, a mono- or disaccharide, an organic compound, an organometallic compound, or an inorganic compound. It can include a living or senescent cell, bacterium, virus, or part thereof. It can include a biologically active molecule such as a hormone, a growth factor, a growth factor, producing virus, a growth factor inhibitor, a growth factor receptor, an anti-inflammatory agent, an antimetabolite, an integrin blocker, or a complete or partial functional insense or antisense gene. It can also include a man-made particle or material, which carries a biologically relevant or active material. An example is a nanoparticle comprising a core with a drug and a coating on the core.

Bioactive materials also can include drugs such as chemical or biological compounds that can have a therapeutic effect on a biological organism. Bioactive materials include those that are especially useful for long-term therapy such as hormonal treatment. Examples include drugs for contraception and hormone replacement therapy, and for the treatment of diseases such as osteoporosis, cancer, epilepsy, Parkinson's disease and pain. Suitable biological materials can include, e.g., anti-inflammatory agents, anti-infective agents (e.g., antibiotics and antiviral agents), analgesics and analgesic combinations, antiasthmatic agents, anticonvulsants, antidepressants, antidiabetic agents, antineoplastics, anticancer agents, antipsychotics, and agents used for cardiovascular diseases such as anti-restenosis and anti-coagulant compounds. Exemplary drugs include, but are not limited to, antiproliferatives such as paclitaxel and rampamycin, everolimus, tacrolimus, des-aspartate angiotensin I, exochelins, nitric oxide, apocynin, gamma-tocopheryl, pleiotrophin, estradiol, heparin, aspirin and HMG-COA reductase inhibitors such as atorvastatin, cerivastatin, fluvastatin, lovastatin, pravastatin, rosuvastatin, simvastatin, etc.

Bioactive materials also can include precursor materials that exhibit the relevant biological activity after being metabolized, broken-down (e.g. cleaving molecular components), or otherwise processed and modified within the body. These can include such precursor materials that might otherwise be considered relatively biologically inert or otherwise not effective for a particular result related to the medical condition to be treated prior to such modification.

Combinations, blends, or other preparations of any of the foregoing examples can be made and still be considered bioactive materials within the intended meaning herein. Aspects of the present invention directed toward bioactive materials can include any or all of the foregoing examples.

The term “medical device” refers to an entity not produced in nature, which performs a function inside or on the surface of the human body. Medical devices include but are not limited to: biomaterials, drug delivery apparatuses, vascular conduits, stents, plates, screws, spinal cages, dental implants, dental fillings, braces, artificial joints, embolic devices, ventricular assist devices, artificial hearts, heart valves, venous filters, staples, clips, sutures, prosthetic meshes, pacemakers, pacemaker leads, defibrillators, neurostimulators, neurostimulator leads, and implantable or external sensors. Medical devices are not limited by size and include micromechanical systems and nanomechanical systems which perform a function in or on the surface of the human body. Embodiments of the invention include such medical devices.

The term “substrate” refers to any physical object that can be submerged in a bath and subjected to electrolytic, electroless or electrophoretic deposition with metal ions or electrolytic, electroless or electrophoretic codeposition with metal ions and anchors.

The terms “implants” or “implantable” refers to a category of medical devices, which are implanted in a patient for some period of time. They can be diagnostic or therapeutic in nature, and long or short term.

The term “self-assembly” refers to a nanofabrication process to form a material or coating, which proceeds spontaneously from a set of ingredients. A common self-assembly process includes the self-assembly of an organic monolayer on a substrate. The process of electroless deposition or codeposition, which continues spontaneously and auto-catalytically from a set of ingredients, can also be considered a self-assembly process.

The term “stents” refers to devices that are used to maintain patency of a body lumen or interstitial tract. There are two categories of stents; those which are balloon expandable (e.g., stainless steel) and those which are self expanding (e.g., nitinol). Stents are currently used in peripheral, coronary, and cerebrovascular vessels, the alimentary, hepatobiliary, and urologic systems, the liver parenchyma (e.g., porto-systemic shunts), and the spine (e.g., fusion cages). In the future, stents will be used in smaller vessels (currently minimum stent diameters are limited to about 2 millimeters). For example, they will be used in the interstitium to create conduits between the ventricles of the heart and coronary arteries, or between coronary arteries and coronary veins. In the eye, stents are being developed for the Canal of Schlem to treat glaucoma.

The phrase “composite structure” as used herein refers to the material overlying a substrate that results from an electrochemical deposition process that does not include any bioactive materials.

The phrase “bioactive composite structure” as used herein refers to the material overlying a substrate that includes bioactive materials.

The phrase “electrochemical process” as used herein means an electrolytic (also known as electroplating), electroless or electrophoretic deposition or codeposition process. A deposition process refers to deposition of metal alone through an electrolytic, electroless or electrophoretic process (although, as will be understood by one of skill in the art, electroless and electrophoretic processes also involve ions of a reducing agent). A codeposition process refers to approximately concurrent deposition of metal and particles of a bioactive material-containing coating through an electrolytic, electroless or electrophoretic process. Again, the anchors that are codeposited through an electrochemical process can, but need not, include bioactive materials. If the codeposited anchors do contain bioactive materials, after an electrochemical codeposition process, the formed structure would be a bioactive composite structure. If the codeposited anchors do not contain bioactive materials, after an electrochemical codeposition process, the formed structure would be a composite structure.

The term “solution” as used herein means any liquid in which an electrochemical process takes place and can be, without limitation, an electrolyte solution, an electrochemical solution and an electroless or electrophoretic bath.

The phrase “substantially similar characteristics” means physical characteristics that are more similar than the physical characteristics between a chosen bioactive material-containing coating and the metal of a substrate.

II. Description of Figures

U.S. Pat. Nos. 5,292,331 and 5,135,536 to Boneau and Hilstead respectively, and the references cited therein, make it clear that stents can be configured and constructed in many different ways. The present invention is applicable to all known stent configurations, and can be applied to any type of stent construction.

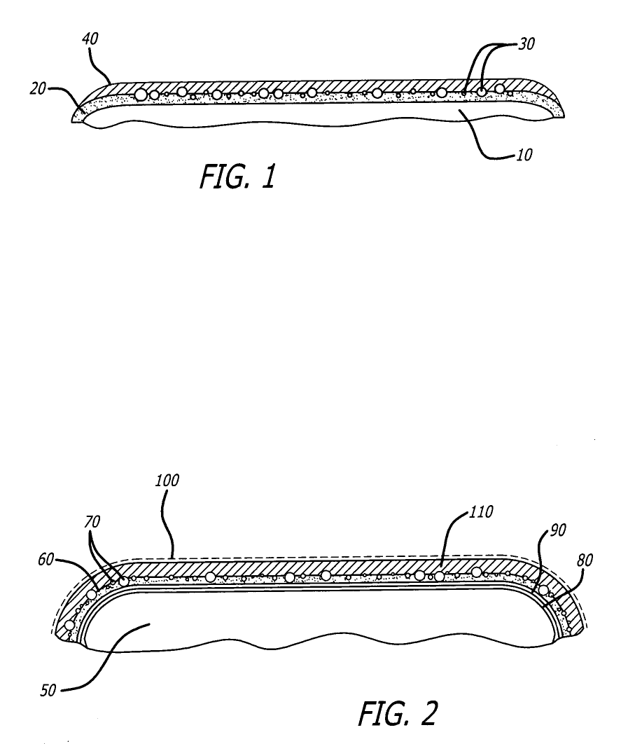

FIG. 1 depicts a schematic representation of a cross section of a stent 10 of the present invention. The stent 10 representationally depicted in FIG. 1 includes a metallic layer 20 formed through an electrochemical process. Anchors 30 are codeposited into the forming metallic layer 20 during the electrochemical process. After forming the metallic layer 20 with codeposited anchors 30, a bioactive material-containing coating 40 is adhered to the surface of the metallic layer 20 and anchors 30.

In one embodiment of the methods of the present invention, the metallic layer, anchors and the adhered bioactive material-containing coating are applied to the entire surface of the stent. In another embodiment of the present invention, the metallic layer, anchors and adhered bioactive material coating are only applied to portions of the stent. When the metallic layer, anchors and the bioactive material-containing coating are only applied to portions of the stent, the stent can be masked in portions that will not include these components. For instance, these portions of the stent can be masked with a material such as, without limitation, Miccrostop® (Michigan Chrome & Chemical Corp., Detroit, Mich.) polyesters, acrylic, wax, etc. Application of the mask can be followed by removing the mask preferentially from the surface of the stent using a laser, sandblaster, or other appropriate methods. Any pattern can be made by selectively removing mask material. A metallic layer with codeposited anchors can then be codeposited onto portions of the stent where mask material was removed followed by adherence of the bioactive material-containing coating to areas of the stent that include the metallic layer and anchors.

FIG. 2 depicts a cross section of a stent 50 with a metallic layer 60 formed through an electrochemical process. Anchors 70 are codeposited into the forming metallic layer 60 during the electrochemical process. After forming the metallic layer 60 with codeposited anchors 70, a bioactive material-containing coating 110 is adhered to the surface of the metallic layer 60 and anchors 70. After adhering the bioactive material-containing coating 110 to the surface of the metallic layer 60 and anchors 70, a bioactive composite structure is formed (assuming, in this example, that the anchors 70 do not also contain bioactive materials; if the anchors did contain bioactive materials, a bioactive composite structure would exist before the bioactive material containing-coating is adhered to the surface of the metallic layer 60 and anchors 70). Again, the bioactive material-containing coating can be adhered to the entire surface of the stent 50 or to one or more discrete portions of the stent 50. The embodiment of the stents of the present invention depicted in FIG. 2 also includes a strike layer 80 (described more fully below), a seed layer 90 (described more fully below) and a topcoat 100 (described more fully below).

III. Methods of Manufacture

Embodiments of the invention include methods of coating substrates including implantable medical devices with bioactive materials to form bioactive composite structures with enhanced adhesion characteristics.

A. Substrate and Substrate Preparation

The substrates of the present invention can be prepared in any suitable manner prior to forming a composite or bioactive composite structure on its surface. For example, the substrate surface can be sensitized and/or catalyzed prior to performing electroless or electrophoretic codeposition processes (if the surface of the substrate is not itself autocatalytic). Metals such as tin (Sn) can be used as sensitizing agents. Many metals (e.g., nickel [Ni], cobalt [Co], copper [Cu], silver [Ag], gold [Au], palladium [Pd], platinum [Pt]) are good auto catalysts. Palladium, Pt, and Cu are examples of “universal” nucleation center forming catalysts. In addition, many non-metals are good catalysts as well.

Before creation of a metallic layer with codeposited anchors, the substrate also can be rinsed and/or precleaned if desired. Any suitable rinsing or pre-cleaning liquid or gas could be used to remove impurities from the surface of the substrate before creating the metallic layer with codeposited anchors. Also, in some embodiments involving electroless or electrophoretic codeposition, distilled water can be used to rinse the substrate after sensitizing and/or catalyzing, but before performing the electroless or electrophoretic process in order to remove loosely attached molecules of the sensitizer and/or catalyst.

Prior to creating the metallic layer with codeposited anchors, the substrates of the present invention also can undergo an anodic process. In this process, the substrate is submerged in a hydrochloric acid bath. Current is passed through the hydrochloric acid bath, creating small pits in the substrate. Such pits promote adhesion. Also, a sensitizing agent and/or catalyst can be deposited on the substrate to assist in the creation of nucleation centers leading to the formation of the composite or bioactive composite structure. Loosely adhered nucleation centers can also be removed from the surface of the substrate using, for example, a rinsing process.

A substrate also can be immersed in a “striking” bath as described in U.S. application Ser. No. 10/701,262 filed on Nov. 3, 2003 which is hereby incorporated by reference for all it contains regarding striking baths. Specifically, in a striking bath, a current is applied across the substrate causing metal ions to move to the device and plate the surface. This step causes an intermediate or “strike” layer to be formed on the surface of the substrate. Metal ions for this first striking bath are chosen to be compatible with the material making up the substrate itself. For example, if the underlying substrate is made of cobalt chrome, cobalt ions are used. It has been found that this strike layer improves overall adherence of the composite or bioactive composite structure to the substrate as well as increasing the rate of deposition during subsequent electrochemical processing. In one embodiment, when striking is performed, the substrate is rinsed with water prior to subsequent electrochemical processing.

Substrates of the present invention also can be immersed in a bath to form a seed layer (also disclosed in co-pending U.S. patent application Ser. No. 10/701,262 filed on Nov. 3, 2003, which is incorporated by reference herein for all it contains regarding seed layers). A seed layer is an electrolessly deposited metallic layer that is deposited before codeposition of metal and anchors. In one embodiment, a seed layer can be formed directly onto the surface of a substrate. In another embodiment, a seed layer can be formed on the surface of a strike layer. Metals for this seed layer also are chosen to be compatible with the material making up the substrate itself and/or the strike layer. A seed layer can be beneficial because it also can enhance the deposition and adhesion of subsequently deposited composite or bioactive composite structures. In one embodiment, when a seed layer is formed, the substrate is rinsed with water prior to subsequent electroless and/or electrophoretic deposition or codeposition.

B. Electrochemical Processes

After a substrate has been prepared according to any of the treatments described above, the substrate undergoes an electrochemical codeposition process to create a metallic layer with anchors comprised of the same coating material (or a material with substantially similar characteristics) that will later be adhered to the substrate. In electrolytic deposition, an anode and cathode are electrically coupled through an electrolyte. As current passes between the electrodes, metal is deposited on the cathode while it is either dissolved from the anode or originates from the electrolyte solution. Electrolytic deposition processes are well known in, for example, the metal plating industry and in the electronics industry.

An exemplary reaction sequence for the reduction of metal in an electrolytic deposition process is as follows:
M^{Z+}ₛₒₗᵤₜᵢₒₙ+Z^{e}→M_{lattice (electrode)}
In this equation, M is a metal atom, M^{Z+} is a metal ion with z charge units and e is an electron (carrying a unit charge). The reaction at the cathode is a reduction reaction and is the location where electrolytic deposition occurs. There is also an anode where oxidation takes place. To complete the circuit, an electrolyte solution is provided. The oxidation and reduction reactions occur in separate locations in the solution. In an electrolytic deposition process, the substrate is a conductor as it serves as the cathode in the process. Specific electrolytic deposition conditions such as the current density and metal ion concentration can be determined by those of ordinary skill in the art.

Electroless deposition processes can also be used in accordance with the methods of the present invention. In an electroless deposition process, current does not pass through a solution. Rather, the oxidation and reduction processes both occur at the same “electrode” (i.e., on the substrate). It is for this reason that electroless deposition results in the deposition of a metal and an anodic product (e.g., nickel and nickel-phosphorus).

In an electroless deposition process, the fundamental reaction is:
M^{Z+}ₛₒₗᵤₜᵢₒₙ+R_{ed solution}→M_{lattice (catalytic surface)}+OXₛₒₗᵤₜᵢₒₙ
In this equation, R is a reducing agent, which passes electrons to the substrate and the metal ions. Ox is the oxidized byproduct of the reaction. In an electroless process, electron transfer occurs at substrate reaction sites (initially the nucleation sites on the substrate; these then form into sites that are tens of nanometers in size). The reaction is first catalyzed by the substrate and is subsequently auto-catalyzed by the reduced metal as a metal matrix forms.

The present invention also provides for electrophoretic deposition or codeposition methods. In electrophoretic deposition or codeposition methods, a slight charge is placed onto the substrate to be coated in order to attract positively-charged metal ions and/or positively-charged anchors. The amount of charge placed onto the substrate is not, however, sufficient to change the balance of the process into an electrolytic deposition (or electrolytic codeposition) only process as described above. Thus, the reactions occurring in the bath resemble electroless processes but with a migration of positively-charged materials toward the slightly-charged substrate.

The electroless and electrophoretic codeposition baths of the present invention comprise at least metal ions, a reducing agent and anchors. The solvent that is used in the e lectroless deposition bath can include water so that the deposition bath is aqueous. Deposition conditions such as the pH, deposition time, bath constituents, and deposition temperature can be chosen by those of ordinary skill in the art.

Any suitable source of metal ions can be used in the methods of the present invention. The metal ions in the bath can be derived from soluble metal salts before they are in the bath. In solution, the ions forming the metal salts can dissociate from each other. Non-limiting examples of suitable metal salts for nickel ions include nickel sulfate, nickel chloride, and nickel sulfamate. Non-limiting examples of suitable metal salts for copper ions include cupric and cuprous salts such as cuprous chloride or sulfate. Non-limiting examples of suitable metal salts for tin cations can include stannous chloride or stannous floroborate. Other suitable salts useful for depositing other metals are known in the electroless and electrophoretic deposition art. Different types of salts can be used if a metal alloy matrix is to be formed.

Reducing agents reduce the oxidation state of the metal ions in solution so that the metal ions deposit on the surface of the substrate as metal. Exemplary reducing compounds that can be used in accordance with the present invention include, without limitation, boron compounds such as amine borane and phosphites such as sodium hypophosphite. The amount of the reducing agent used generally is not critical. In one embodiment, the reducing agent can be included in the range of about 0.05 to about 0.5 mole/liter. In another embodiment, the reducing agent can be included in the range of about 0.15 to about 0.3 mole/liter.

Suitable anchors and bioactive material-containing coatings include, without limitation, a variety of polymers. Suitable polymers that can be used include soluble and insoluble, biodegradable and nonbiodegradable polymers. These can be, without limitation, hydrogels or thermoplastics, homopolymers, copolymers or blends, natural or synthetic.

Rapidly bioerodible polymers such as, without limitation, poly[lactide-co-glycolide], polyanhydrides, and polyorthoesters, whose carboxylic groups are exposed on the external surface as their smooth surface erodes, are excellent candidates for drug delivery systems. In addition, polymers containing labile bonds, such as, without limitation, polyanhydrides and polyesters, are well known for their hydrolytic reactivity. Their hydrolytic degradation rates can generally be altered by simple changes in the polymer backbone.

Representative natural polymers that can be used as anchors and bioactive material-containing coatings include, without limitation, proteins, such as zein, modified zein, casein, gelatin, gluten, serum albumin, or collagen, and polysaccharides, such as, without limitation, cellulose, dextrans, polyhyaluronic acid, polymers of acrylic and methacrylic esters and alginic acid. Representative synthetic polymers that can be used in accordance with the present invention include, without limitation, polyphosphazines, poly(vinyl alcohols), polyamides, polycarbonates, polyalkylenes, polyacrylamides, polyalkylene glycols, polyalkylene oxides, polyalkylene terephthalates, polyvinyl ethers, polyvinyl esters, polyvinyl halides, polyvinylpyrrolidone, polyglycolides, polysiloxanes, polyurethanes and copolymers thereof. Synthetically modified natural polymers that can be used in accordance with the present invention include, without limitation, alkyl celluloses, hydroxyalkyl celluloses, cellulose ethers, cellulose esters, and nitrocelluloses. Other polymers that can be used in accordance with the present invention include, but are not limited to, methyl cellulose, ethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, carboxymethyl cellulose, cellulose triacetate, cellulose sulfate sodium salt, poly(methyl methacrylate), poly(ethyl methacrylate), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate) polyethylene, polypropylene, poly(ethylene glycol), poly(ethylene oxide), poly (ethylene terephthalate), poly(vinyl acetate), polyvinyl chloride, polystyrene, polyvinyl pyrrolidone, and polyvinylphenol. Representative bioerodible polymers include polylactides, polyglycolides and copolymers thereof, poly(ethylene terephthalate), poly(butic acid), poly(valeric acid), poly(lactide-co-caprolactone), poly[lactide-co-glycolide], polyanhydrides, polyorthoesters, blends and copolymers thereof.

These described polymers can be obtained from sources such as Sigma Chemical Co., St. Louis, Mo., Polysciences, Warrenton, Pa., Aldrich, Milwaukee, Wis., Fluka, Ronkonkoma, N.Y., and BioRad, Richmond, Calif. or else synthesized from monomers obtained from these suppliers using standard techniques.

Suitable anchors can also be formed from non-polymeric materials including, without limitation, metals and ceramics.

In the electrophoretic codeposition methods of the present invention, anchors can be given a positive charge by coupling a surfactant to the anchors. Non-limiting examples of cationic surfactants that can be used in accordance with the present invention include hexadecyl trimethyl ammonium bromide (HTAB), benzethonium chloride (BZTC) and cationic cyclodextrin complexes such as, without limitation, N, N-diethylaminoethyl-β-cyclodextrin and 2,3-di-(N,N-diethylaminoethyl)-N-amino-2,3-deoxy-β-cyclodextrin. A suitable example of a zwitterionic surfactant that can be used in accordance with the present invention includes, without limitation 3-[(3-cholamido-propyl)-dimethyl-ammonio]-1-propanesulfonate (CHAPS).

Dispersing agents also can be used in accordance with the present invention. Anionic dispersing agents that can be used in accordance with the present invention include sodium lignosulfonate, sodium naphthalene sulfonate-formaldehyde condensate (“Lomar D”), sodium polystyrene sulfonate (“Flexan 130”), polyacrylic acid (Acumer 9400 and Good-Rite K-732) and organic phosphate ester (Emphos CS-1361). Nonionic dispersing agents that can be used in accordance with the present invention include, without limitation, aliphatic alcohol ethoxylate (Atlas G5000), ethylene oxide-propylene oxide block copolymer (HLB=17.0; Pluronic P65) and polyoxyethylene (20) monolaurate (HLB=16.7; Tween 20™). Cationic dispersing agents that can be used in accordance with the present invention include, without limitation, dimethyl dicoco ammonium chloride (Arquad® 2C-75, Akzona Inc., Enka, N.C.) and N-alkyl(soya)trimethyl ammonium chloride (Arquad® S-50, Akzona Inc., Enka, N.C.). A zwitterionic dispersing agent that can be used in accordance with the present invention includes, without limitation, palmitamidopropylbetaine (Scheercotaine PAB).

Wetting agents also can be used in accordance with the present invention. Anionic wetting agents that can be used in accordance with the present invention include, without limitation, sodium lauryl sulfate, sodium dioctyl sulfosuccinate (“aerosol otb”), sodiumdodecyl benzene sulfonate (“witconate 90”) and sodium isopropyl naphthalene sulfonate (“aerosol OS”). Nonionic wetting agents that can be used in accordance with the present invention include, without limitation, secondary alcohol ethoxylate (“tergitol® 15-5-5”; Union Carbide Chemicals & Plastics Technology Corp., Danbury, Conn.) and pluronic L 62 (a block copolymer of propylene oxide and ethylene oxide).

During the electrochemical codeposition processes of the present invention, metal ions deposit over the surface of the substrate. Without being bound by theory, it is believed that tens of nanometers of metal deposit onto the surface of the substrate. Following the deposition of tens of nanometers of metal, anchors begin to codeposit with the metal. Thus, the anchors and the metal atoms can deposit substantially simultaneously. When codepositing metal atoms and anchors, the anchors are incorporated into the metal matrix. The forming metallic layer confines the anchors within the formed composite or bioactive composite structure.

By codepositing the anchors along with the metal, the concentration of the anchors in the composite or bioactive composite structure can be high. Moreover, potential problems associated with impregnating porous structures with anchors are not present in the electrochemical codeposition methods of the present invention.

As an example of the methods of the present invention, a nickel-phosphorous alloy matrix can be electrolessly codeposited on a substrate along with anchors. In one embodiment, the substrate can be activated and/or catalyzed (using, e.g., Sn and/or Pd) prior to metallizing. To produce the alloy matrix, the electroless deposition bath can contain NiSO₄ (26 g/L), NaH₂PO₂ (26 g/L), Na-acetate (34 g/L) and malic acid (21 g/L). The bath can contain ions derived from the previously mentioned salts. Anchors also are in the bath. Non-limiting examples of anchors that can be included in the presently-described bath include 1500 mg/L polylactide, 1500 mg/L polyglycolide, and/or 1500 mg/L polystyrene. In this embodiment, sodium hypophosphite is the reducing agent and nickel ions are reduced by the sodium hypophosphite. The temperature of the bath is from about room temperature to about 95° C. depending on desired deposition time. The pH is generally from about 5 to about 7 (these processing conditions could be used in other embodiments). The substrate to be coated is then immersed in the bath and a composite structure is formed on the exposed surface of the substrate after a predetermined amount of time. The nickel ions in solution deposit onto the exposed surface of the substrate as pure nickel (reduction reaction) along with nickel-phosphorous alloy (oxidation reaction); the anchors codeposit along the crystallite and grain boundaries of the deposited metal matrix to form a composite structure. Typically, the amount of phosphorous ranges from about greater than 1% to about less than 25% (mole %) and can be varied by techniques known to those skilled in the art.

The bath also can include complexing agents, stabilizers, and buffers. Complexing agents are used to hold the metal in solution. Buffers and stabilizers are used to increase bath life and improve the stability of the bath. Buffers are used to control the pH of the bath. Stabilizers can be used to keep the solution homogeneous. Exemplary stabilizers include lead, cadmium, copper ions, etc. Complexing agents, stabilizers and buffers are well known in the electrochemical deposition art and can be chosen by those of ordinary skill in the art.

The metallic matrix of the composite structure formed during the electrochemical codeposition methods of the present invention can include any suitable metal. The metal in the metallic matrix can be the same as or different from the substrate metal (if the substrate is metallic). The metallic matrix can include, for example, noble metals or transition metals. Suitable metals include nickel, copper, cobalt, palladium, platinum, chromium, iron, gold, and silver and alloys thereof. Examples of suitable nickel-based alloys include nickel-chromium, nickel-phosphorous, and nickel boron. Any of these or other metallic materials can be deposited using a suitable electrochemical codeposition process. Appropriate metal salts can be selected to provide appropriate metal ions in the bath for the metal matrix that is to be formed.

After contacting a solution or bath, a composite or biocomposite structure has been formed on the substrate using an electrochemical codeposition process. After forming the composite or bioactive composite structure, the structure/substrate combination is removed from the solution or bath and subjected to subsequent processing.

C. Subsequent Processing

After electrochemical codeposition onto the surface of the substrate, the device can be processed further to alter its clinical features.

The composite or bioactive composite structures formed by the described electrochemical methods include anchors that are exposed on the surface of the deposited metallic layer. A material that is compatible with these anchors (i.e. because it is the same material or has substantially similar physical characteristics) can be applied to the surface of the implantable medical device.

1. Adherence of Bioactive Material-Containing Coating to Anchors

In one embodiment, the anchors and the applied bioactive material-containing coating can be sufficiently non-inflammatory and biocompatible so that inflammatory responses do not prevent the delivery of the bioactive materials to tissue. In another embodiment, the anchors and the applied bioactive material-containing coating can be sufficiently porous to permit efflux of the bioactive materials. In yet another embodiment, the anchors and the applied bioactive material-containing coating can provide at least partial protection of the biologically active molecules from adverse effects of proteases and nucleases.

The applied bioactive material-containing coatings can be adhered to the surface of a substrate containing anchors by a variety of techniques that are well-known to those of ordinary skill in the art. For instance, the bioactive material-containing coating can be applied by dip coating, spray coating, roll coating, vapor deposition, etc. These techniques are generally known to those of ordinary skill in the art. Spray coating in particular is recently described in U.S. Pat. Nos. 6,861,088 and 6,743,463, both to Weber et al., which are hereby incorporated by reference for all they contain regarding spray coating.

2. Topcoat Formation

If desired, a topcoat can be formed on the bioactive composite structures of the present invention. The topcoat can include any suitable material and can be in any suitable form. It can be amorphous or crystalline, and can include a metal, ceramic, etc. The topcoat can also be porous or solid (continuous).

The topcoat can be deposited using any suitable process. For example, the topcoat can be formed by processes such as, without limitation, dip coating, spray coating, roll coating, vapor deposition, etc.

In some embodiments, the topcoat can improve the properties of the bioactive composite structure. For example, the topcoat can include a membrane (e.g., collagen type 4) that is covalently bound to the bioactive composite structure. The topcoat's function can be to induce endothelial attachment to the surface of a bioactive composite structure, while the bioactive material in the bioactive composite structure diffuses from below the topcoat. In another embodiment, a growth factor such as endothelial growth factor (EGF) or vascular endothelial growth factor (VEGF) is present in a topcoat that is on a bioactive composite structure. The growth factor is released from the topcoat to induce endothelial growth while the bioactive composite structure releases an inhibitor of smooth muscle cell growth.

In yet another embodiment of the present invention, the topcoat can improve the radiopacity of a medical device which includes the bioactive composite structure, while the underlying bioactive composite structure releases molecules to perform another function. For example, drugs can be released from the bioactive composite structure to prevent smooth muscle cell overgrowth, while a topcoat on the bioactive composite structure improves the radiopacity of the formed medical device.

The topcoat can also be used to alter the release kinetics of the bioactive material in the underlying bioactive composite structure. For example, a topcoat could require the bioactive material contained in the bioactive material-containing coating to travel through an additional layer of material before entering the surrounding environment, thereby delaying the release of the bioactive material. The release kinetics of the formed medical device can be adjusted in this manner.

Although medical devices such as stents are discussed in detail, it is understood that embodiments of the invention are not limited to stents or for that matter, to macroscopic devices. For example, embodiments of the invention could be used in any device or material, regardless of size and includes artificial hearts, plates, screws, mems (microelectromechanical systems), and nanoparticle based materials and systems, etc. Further, the substrate can be porous or solid, flexible or rigid, and can have a planar or non-planar surface (e.g., curved).

The terms and expressions which have been employed herein are used as terms of description and not of limitation, and there is no intention in the use of such terms and expressions of excluding equivalents of the features shown and described, or portions thereof, it being recognized that various modifications are possible within the scope of the invention claimed. Moreover, any one or more features of any embodiment of the invention can be combined with any one or more other features of any other embodiment of the invention, without departing from the scope of the invention.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a” and “an” and “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventors for carrying out the invention. Of course, variations on those preferred embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed is:

1. A method comprising:
providing a solution comprising metal ions and anchors;
contacting a substrate with said solution thereby forming a metallic composite structure through an electrochemical process wherein at least a subset of said anchors are exposed on at least a portion of the surface of said structure; and
adhering a bioactive material-containing coating to said surface of said structure and said exposed anchors wherein said bioactive material-containing coating and said anchors have physical characteristics that are more similar than the physical characteristics of said bioactive material coating and said substrate.

2. The method according to claim 1, wherein said anchors include a bioactive material and said formed structure is a bioactive composite structure.

3. The method according to claim 1, wherein said anchors are free of bioactive materials and said formed structure is a composite structure.

4. The method according to claim 1, wherein said electrochemical process is selected from the group consisting of an electrolytic codeposition process, an electroless codeposition process and an electrophoretic codeposition process.

5. The method according to claim 1, wherein said anchors comprise a polymer.

6. The method according to claim 1, wherein said bioactive material-containing coating comprises a polymer.

7. The method according to claim 1, wherein said anchors and said bioactive material-containing coating both comprise a polymer.

8. The method according to claim 1, wherein said anchors and said bioactive material-containing coating are only applied to a portion of substrate.

9. The method of claim 1, wherein said substrate is a stent.

10. The method of claim 1, further comprising forming a topcoat over said adhered bioactive material-containing coating.

11. A medical device comprising:
a substrate having a metallic composite structure containing anchors wherein said structure is formed through an electrochemical process wherein at least a subset of said anchors are exposed on the surface of said structure; and
a bioactive material-containing coating adhered to said surface of said structure and said exposed anchors wherein said bioactive material-containing coating and said anchors have physical characteristics that are more similar than the physical characteristics of said bioactive material coating and said substrate.

12. The medical device according to claim 11, wherein said anchors include a bioactive material and said formed structure is a bioactive composite structure.

13. The medical device according to claim 11, wherein said anchors are free of bioactive materials and said formed structure is a composite structure.

14. The medical device according to claim 11, wherein said electrochemical process is selected from the group consisting of an electrolytic codeposition process, an electroless codeposition process and an electrophoretic codeposition process.

15. The medical device according to claim 11, wherein said bioactive material-containing coating comprises a polymer.

16. The medical device according to claim 11, wherein said anchors and said bioactive material-containing coating both comprise a polymer.

17. The medical device according to claim 11, wherein said anchors and said bioactive material-containing coating are only applied to a portion of substrate.

18. The medical device according to claim 11, wherein said substrate is a stent.

19. The medical device according to claim 11, further comprising a topcoat over said adhered bioactive material-containing coating.
